(19) United States
(12) Patent Application Publication
(10) Pub. No.: US 2026/0275632 A1
(43) Pub. Date: Sep. 17, 2026

(54) DISCHARGE ARRANGEMENT FOR DISCHARGING LIGNOCELLULOSIC MATERIAL

(71) Applicant: VALMET AB, Sundsvall (SE)

(72) Inventor: Robert WAHLBERG, Sundsvall (SE)

(73) Assignee: VALMET AB, Sundsvall (SE)

(21) Appl. No.: 19/479,995

(22) PCT Filed: Apr. 15, 2024
(86) PCT No.: PCT/SE2024/050356

§ 371 Date: Oct. 29, 2025

(30) Foreign Application Priority Data
May 3, 2023 (SE) 2330197-1

(51) Int. Cl. D21C 7/08

(52) U.S. Cl. CPC D21C 7/08

(57) ABSTRACT

A discharge arrangement for discharging lignocellulosic material includes: a screw conveyor section including an inlet for receiving lignocellulosic material; an outlet section configured to receive lignocellulosic material from the screw conveyor section; a conveyor screw rotatably arranged within the screw conveyor section for transporting the material in a material transport direction from the inlet to the outlet section; and a material spreader rotatably mounted within the outlet section, wherein the material spreader is rotatable in a first rotational direction and an opposite second rotational direction around a rotational axis extending in the material transport direction, which material spreader includes at least one blade configured to rotate around the rotational axis to transport the lignocellulosic material radially outwards and in the first and second rotational directions during rotation of the material spreader.

### TECHNICAL FIELD

The present invention relates to a discharge arrangement for discharging lignocellulosic material, a discharge system comprising the discharge arrangement, a method for controlling the discharge system and a computer program product.

### BACKGROUND

Lignocellulosic material or biomass material is abundant and can provide a sustainable resource for producing, for example, fuels, chemicals, and biobased materials. Lignocellulosic material normally comprises primarily cellulose, hemicellulose, and lignin. The removal of hemicellulose is a key step of the pulp production process and may be carried out in a steam or water pre-hydrolysis stage. Pre-hydrolysis is sometimes called hydro-thermal treatment or just pre-treatment since it is a treatment step before further treatment, e.g., enzymatic hydrolysis. Pre-hydrolysis is carried out at milder conditions compared to a hydrolysis process and is used for hydrolyzing the hemicellulose content of the biomass. A reactor used for pre-hydrolysis of the lignocellulosic material are usually either horizontal or vertical.

A discharge arrangement may be arranged to receive the material from the reactor for transport to subsequent processing steps, e.g., a steam explosion step. In prior art reactors, a large amount of steam is needed to discharge the often wet and heavy material through the discharge arrangement. This results in a high steam consumption in the reactor. It is anticipated that a more effective outfeed from the reactor could reduce the steam consumption in the reactor.

WO2020/091675 relates to a discharge arrangement with reduced steam consumption. The discharge arrangement comprises a discharge section arranged to receive lignocellulosic material from a reactor. The discharge section accommodates a conveyor screw for transport of the material towards a downstream outlet section, where a material spreader is rotatably arranged to propel the material radially outwards towards an outlet in a side wall of the discharge device. The material spreader may comprise a plurality of blades extending in a radial direction perpendicular to a rotational axis of the material spreader. This arrangement makes it possible to reduce the amount of steam injected at an inlet side of the discharge device to transport the material through the discharge device and through the outlet.

One problem associated with prior art devices is clogging of the discharge arrangement.

### SUMMARY

It is an object of the invention to reduce the risk of clogging of the discharge arrangement.

These and other objects are achieved by the present invention by means of a discharge arrangement, a discharge system, a method, and a computer program product according to the independent claims.

According to a first aspect of the invention, a discharge arrangement is provided. The discharge arrangement comprises a screw conveyor section provided with an inlet for receiving lignocellulosic material (for example from a treatment reactor such as a pre-hydrolysis reactor) and an outlet section arranged to receive lignocellulosic material from the screw conveyor section. The outlet section may be described as arranged downstream of the screw conveyor section (as seen in a longitudinal direction of the discharge arrangement). The outlet section is either integrally formed with, or removably connected to, the screw conveyor section in such a way that lignocellulosic material may be fed from the screw conveyor section to the outlet section. A conveyor screw is rotatably arranged within the screw conveyor section for transporting the material in a material transport direction, which extends substantially coaxially or in parallel with a rotational axis of the conveyor screw, from the inlet to the outlet section. A material spreader is rotatably mounted within the outlet section, which material spreader is rotatable in a first rotational direction and an opposite second rotational direction around a rotational axis extending in the material transport direction. The material spreader may comprise at least one blade, preferably at least two blades, and even more preferably three or four blades, configured to rotate around the rotational axis to transport the lignocellulosic material radially outwards and in the first and second rotational directions during rotation of the material spreader. The discharge arrangement further comprises a drive unit configured to rotate the material spreader around the rotational axis. The discharge arrangement also comprises a first outlet and a second outlet arranged in a side wall of the outlet section, said first and second outlets being arranged at a distance from one another along an inner perimeter of the side wall, said first outlet being arranged to receive material transported by the blade in the first rotational direction and the second outlet being arranged to receive material transported by the blade in the second rotational direction. An outlet may, for example, be an opening or an outlet nozzle in the side wall of the discharge device. An outlet may have a fixed or adjustable size. The outlet section may be described as substantially tubular or cylindrical. The side wall may be described as the mantle or lateral surface of the outlet section. The first and second outlets may be described as arranged peripherally of the material spreader. The first and second outlets may be described as arranged at a distance from one another, or spaced apart, in the circumferential direction of the outlet section.

It has been discovered that material deposits accumulate over time on the leeside surface of a blade that can only be rotated in one direction. The leeside is the side of the blade that faces away from the rotational direction of the blade and does not propel the material in said rotational direction. These material deposits will eventually bring the discharge arrangement to a halt unless they are removed.

The invention is based on the surprising finding that this problem can be overcome by alternating between a first rotational direction and an opposite second rotational direction of the material spreader. Opposite surfaces of the blade will interact with and propel the material towards the outlets, and the material passing over said surfaces will remove material deposits therefrom. This arrangement requires at least two separate outlets, one configured to receive the material when the material spreader is rotated in the first rotational direction and the other configured to receive the material when the material spreader is rotated in the second rotational direction.

Within the context of the present disclosure, it is understood that connected members may be connected by means of one or more intermediate members.

Further, a member arranged to receive a fluid or solid material from another member may receive the fluid or solid material from one or more intermediate members located between said members. It is also understood that upstream and downstream refer to the flow direction of the lignocellulosic material through the discharge arrangement.

Advantageously, the material spreader and the conveyor screw are mounted on separate rotatable axles or shafts rotated by separate drive units. This makes it possible to apply a higher rotational speed to the material spreader relative the rotational speed of the conveyor screw. Separate shafts and drive units also make it possible to rotate the material spreader and the conveyor screw in different directions.

The discharge arrangement may comprise a plug section arranged between the discharge screw section and the outlet section, wherein the density of the material is increased, so that a substantially gas tight plug of material is created that prevents steam from moving through the plug against the material transport direction, so called blow back. The plug makes it possible to maintain the pressure in devices located on opposite sides of the plug section. For this purpose, the plug section may have a narrowing cross-section in the material transport direction. In these embodiments, the material spreader may have the additional function of disintegrating the plug of material to facilitate transport of the material to downstream treatment steps. Each blade may comprise a jagged edge or surface configured to interact with and tear apart the plug.

The various sections of the discharge arrangement may have the same or different diameters. Each section may also have a varying diameter along the longitudinal axis of the discharge arrangement.

Advantageously, the first outlet extends outwards through the side wall of the outlet section substantially along a tangent to the first rotational direction of the material spreader and/or the second outlet extends outwards through the side wall of the outlet section substantially along a tangent to the second rotational direction of the material spreader. The first and/or second outlet may alternatively be described as extending substantially parallel with a tangential direction of the side wall of the outlet section (which outlet section in such an embodiment may be described as substantially tubular/cylindrical). Outlet(s) extending tangentially ensures that the material is propelled by the blade into the first outlet and/or second outlet.

Herein, the tangent to a rotational direction of the material spreader is the forward pointing tangent or tangent vector to the travelling path of the outermost portion of a blade being rotated in the rotational direction of the material spreader. The tangent may be drawn from any point on the travelling path of the outermost portion of the blade.

An outlet may be located at any point on an inner perimeter of the outlet section side wall. Advantageously, the first and second outlets are arranged in a lower portion of the side wall. The first and second outlets may be located on the same or opposite sides of the outlet section seen in a transverse direction of the outlet section. The first and second outlets may also be located on the same or opposite sides of the outlet section seen in the vertical direction of the outlet section.

The at least one blade comprises a first surface configured to transport the material in the first rotational direction and an opposite second surface configured to transport the material in the second rotational direction. That is, the first surface is facing in the first rotational direction, although not necessarily perpendicular to the first rotational direction, and the second surface is facing in the second rotational direction, although not necessarily perpendicular to the second rotational direction.

The blade may extend in a substantially radial direction within a rotational plane orthogonal to the rotational axis of the material spreader.

At least a portion of the first and/or second surfaces may be flat and extend orthogonally to the rotational plane of the blade.

The first and second surfaces may comprise portions that extend in parallel.

At least one, preferably both, of the first and second surfaces of a blade may comprise a portion that extends with an acute angle relative the rotational axis of the material spreader, so that the surfaces diverge from one another seen in the material transport direction. This embodiment is advantageous in that the blade propels the material against the material transport direction and away from a downstream end wall of the outlet section, thus reducing the amount of material deposits on the end wall, and in that the lignocellulosic material moves over larger portions of the surfaces to remove material deposits from said surfaces. In some embodiments, the acute angle may be up to 15 degrees relative the rotational axis of the material spreader.

The material spreader may be arranged so that the blades are positioned near the end wall of the outlet section to scrape of material deposits from the end wall.

One or more blades of the material spreader may be connected to or integrally formed with a rotatable disc arranged to be rotated by the drive unit around the rotational axis of the material spreader.

The discharge arrangement may further comprise a first blow line connected to the first outlet for receiving lignocellulosic material propelled through the first outlet and a second blow line connected to the second outlet for receiving lignocellulosic material propelled through the second outlet.

The discharge arrangement may further comprise a first discharge valve configured to regulate the flow of material through the first outlet and a second discharge valve configured to regulate the flow of material through the second outlet. The first and second discharge valves may, for example, be throttles valves or blow valves. Advantageously, the first and second discharge valves are arranged at or near the first and second outlets, respectively.

The first discharge valve is configured to be (fully or partially) open when the material spreader is rotated in the first rotational direction and the second discharge valve is configured to be (fully or partially) open when the material spreader is rotated in the second rotational direction, thus ensuring that the lignocellulosic material is always propelled through an outlet extending along a tangent to the current rotational direction of the material spreader. The first discharge valve may be configured to be (at least partially) closed when the material spreader is rotated in the second rotational direction and the second discharge valve may be configured to be (at least partially) closed when the material spreader is rotated in the first rotational direction, so that the lignocellulosic material is smoothly discharged from the discharge device through one outlet at a time.

The discharge of lignocellulosic material can be hot or cold depending on the requirements in subsequent steps. A hot discharge involves a steam explosion step, wherein the lignocellulosic material treated with hot steam under pressure is exposed to a sudden decompression, thus generating disintegration of the biomass into small particles. A hot discharge may be beneficial, e.g., in a subsequent saccharification step since the enzymes used in such a step are given excellent access to the lignocellulosic material. Cold blow, or dilution discharge, is applied when the objective is to separate the sugars dissolved in the liquid phase from the remaining solid biomass. It gives the possibility to treat the sugars dissolved during the pre-hydrolysis, mainly from hemicelluloses, separately. Separation of the liquid from the biomass is done in fiber washing equipment.

The first and second discharge valves may be configured to create steam explosions. The first and second discharge valves may also be configured for cold discharge of the lignocellulosic material.

The blow lines are arranged to transport the lignocellulosic material to one or more downstream devices, for example a cyclone or a blow tank, for further treatment of the lignocellulosic material. The blow lines may merge before they reach the next device or may be separately connected to said device.

The discharge arrangement may also comprise a first steam line configured to transport steam from a steam source towards the first outlet and/or a second steam line configured to transport steam from a steam source towards the second outlet. Suitably, the first steam line is connected to the first blow line and the second steam line to the second blow line, so that steam is transported to the outlets through the first and second blow lines.

The discharge arrangement may comprise at least one steam valve configured to control the flow of steam through the first outlet and/or at least one steam valve configured to control the flow of steam through the second outlet.

One or more steam valves may be arranged in the first and/or second steam lines.

The blow line may be provided with one or more steam valves configured to regulate the steam flow through the blow lines. At least one, or each, of the blow lines may be provided with steam valve(s) configured to direct a larger portion of the steam flow, suitably about 85-95 % of the steam, into the outlet section and a smaller portion of the steam flow, suitably 5 -15 %, in a direction away from the outlet section to preheat a downstream portion of the blow line before a change in rotational direction of the material spreader.

The first and second steam lines may be connected to different steam sources. In other embodiments, the first and second steam lines may be connected to the same steam source, in which case the first and second steam lines either merge before the steam source or are separately connected to the steam source.

In this context, a steam source is any suitable part of a plant that may provide steam suitable for this purpose.

According to a second aspect of the invention, there is provided a discharge system comprising a discharge arrangement according to the first aspect of the invention or embodiments thereof and a control unit configured to control the drive unit to alternate the rotational direction of the material spreader between the first and second rotational directions, so that the material spreader is sometimes rotated in the first rotational direction to propel material through the first outlet, and sometimes is rotated in the second rotational direction to propel lignocellulosic material through the second outlet. The control unit may be a single control device or comprise a plurality of control devices configured to communicate with one another, wherein each control device is configured to control one or more parts of the discharge system.

The above-described discharge valves may be controlled by means of a control unit, preferably the same control unit that controls the drive unit rotating the material spreader. This arrangement makes it easy to synchronize the opening and closing of the discharge valves with changes of the rotational direction of the material spreader.

The above-described steam valves may be controlled by the control unit so that steam is injected through the first outlet when the material spreader is rotated in the second rotational direction and through the second outlet when the material spreader is rotated in the first rotational direction. Thus, injection of steam through one outlet contributes to propelling the lignocellulosic material out from the discharge device through the other outlet. The flow of steam through the outlets also keeps the outlets clean.

According to a third aspect of the invention, there is provided a method for controlling the discharge system according to the invention.

The method comprises the steps of transporting the lignocellulosic material towards the material spreader by means of the conveyor screw and controlling the drive unit, for example by means of the control unit, to alternately rotate the material spreader in the first and second rotational directions, so that said lignocellulosic material is alternately propelled out of the discharge device through the first and second outlets. The rotational direction of the control unit may be changed manually or by means of a control unit according to predetermined settings or based on continuously received input data. As explained above, these steps ensure that material deposits on both sides of the blade(s) are removed during use to prevent clogging of the discharge system.

The method may comprise the steps of opening the first discharge valve when the material spreader is rotated in the first rotational direction and open the second outlet when the material spreader is rotated in the second rotational direction. The method may also comprise the steps of closing the first discharge valve when the material spreader is rotated in the second rotational direction and closing the second discharge valve when the material spreader is rotated in the first rotational direction. Advantageously, these steps are carried out automatically by means of the control unit based on continuously received input data or according to predetermined settings.

The method may also comprise the steps of using the control unit to control at least one steam valve configured to control a flow of steam through the first outlet and at least one steam valve configured to control a flow of steam through the second outlet, so that steam is injected through the first outlet when the material spreader is rotated in the second rotational direction and through the second outlet when the material steam is rotated in the first rotational direction. One or more steam valves for regulating the flow through the first outlet may be arranged in a first steam line connected to the first blow line. One or more steam valves for regulating the flow through the second outlet may be arranged in a second steam line connected to the second blow line. Advantageously, these steps are carried out automatically by means of the control unit based on continuously received input data or according to predetermined settings.

Advantageously, the control unit is configured to change the rotational direction of the material spreader with a time interval between one hour and four days depending on one or more process parameters, such as the type of lignocellulosic material. Control of the valves should be synchronized with changes of the rotational direction of the material spreader. The time interval is chosen so that the amount of lignocellulosic material deposited on the surfaces of the blade does not become too large and negatively affects or stops the process.

The control unit may be configured to receive data from at least one measuring unit configured to measure a parameter indicative of the amount of lignocellulosic material deposited on the blade(s). The at least one measuring unit is a part of the discharge arrangement and may, e.g., be a vibration sensor, arranged to measure the vibration of the discharge arrangement or any suitable part thereof. The control unit is then configured to control the rotational direction of the material spreader and other parts of the discharge system in response to the data received from the measuring unit.

According to a fourth aspect of the invention, there is provided a computer program product comprising instructions which, when executed in a processor of the discharge system according to the invention causes the control unit to perform the method according to the third aspect of the invention. The processor may comprise or be comprised by the control unit.

The computer program product allows remote or automated control of the discharge system.

According to a fifth aspect of the invention, there is provided a treatment system comprising a treatment reactor and a discharge arrangement according to the first aspect of the invention or embodiments thereof, wherein the inlet of the screw conveyor section is arranged to receive lignocellulosic material from an outlet of the treatment reactor. The treatment reactor may be configured for hydrothermal treatment/pre-hydrolysis of the lignocellulosic material, for example by being provided with one or more steam inlets for providing steam to heat the lignocellulosic material.

The treatment reactor may comprise a reactor vessel and an internal conveyor screw rotatably arranged in said reactor vessel, the internal conveyor screw being configured to mechanically transport the lignocellulosic material through the reactor vessel in a conveying direction along a rotational axis of the internal conveyor screw and out through the outlet, the outlet being disposed at a downstream end of the reactor vessel.

The reactor vessel may be a substantially horizontally or vertically arranged reactor vessel, wherein the discharge screw arrangement is located vertically below the reactor vessel at the downstream end of the reactor vessel, such that the lignocellulosic material can fall down vertically from the outlet of the reactor vessel into inlet of the discharge screw arrangement.

Alternatively, the reactor vessel may be a substantially vertically arranged reactor vessel, wherein the inlet of the screw conveyor section is arranged substantially in line horizontally with an outlet of the reactor vessel.

According to a sixth aspect of the invention, there is provided a treatment system corresponding to the discharge arrangement according to the first aspect of the invention (or corresponding to the discharge system according to the second aspect of the invention) except that the screw conveyor section is replaced with a substantially horizontally arranged treatment reactor vessel/chamber provided with an inlet for receiving lignocellulosic material, wherein a conveyor screw is rotatably arranged within the reactor vessel for transporting the lignocellulosic material in a material transport direction from the inlet to the outlet section. The reactor vessel is configured for hydrothermal treatment/pre-hydrolysis of the lignocellulosic material, for example by being provided with one or more steam inlets for providing steam to heat the lignocellulosic material.

The features of the embodiments described above are combinable in any practically realizable way to form embodiments having combinations of these features. Further, all features and advantages of embodiments described above with reference to the first aspect of the invention may be applied in corresponding embodiments of the system according to the second, third, fourth, fifth and sixth aspects of the invention and vice versa. In particular, it is noted that the above-described features of embodiments of the first and second aspects of the invention may be applied in corresponding embodiments of the system according to the sixth aspect of the invention, where it is understood that the reactor vessel/chamber corresponds to the screw conveyor section.

### BRIEF DESCRIPTION OF THE DRAWINGS

The invention, together with further objects and advantages thereof, can best be understood by reference to the following description taken together with the accompanying drawings, in which:

FIG. 1 is a schematic illustration of an embodiment of a discharge system according to the invention comprising an embodiment of a discharge arrangement according to the invention;

FIG. 2a is a cross-sectional view of the discharge arrangement shown in figure along line B-B in FIG. 1;

FIG. 2b schematically illustrates an alternative embodiment of the outlet section of the discharge arrangement in FIG. 2a;

FIG. 2c schematically illustrates an alternative embodiment of the outlet section in FIG. 2a and b;

FIG. 3 is a schematic illustration of an embodiment of a discharge system according to the invention;

FIG. 4 is a schematic illustration of an alternative embodiment of a discharge system according to the invention;

FIG. 5 is a schematic illustration of the material spreader of the discharge arrangement in FIG. 1; and

FIG. 6 is a schematic illustration of an alternative embodiment of a material spreader of a discharge system according to the invention.

### DETAILED DESCRIPTION

In the drawings, the same reference designations are used for similar or corresponding elements.

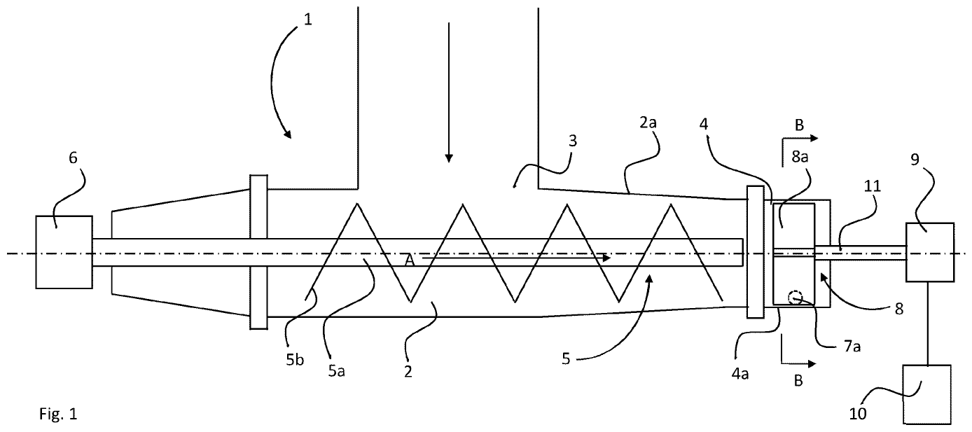

FIG. 1 is a schematic illustration of an embodiment of a discharge system according to the invention which comprises a discharge arrangement 1 and a control unit 10. The discharge arrangement 1 comprises a screw conveyor section 2 and an outlet section 4. The screw conveyor section 2 is arranged at an upstream end of the discharge arrangement 1 and is provided with an inlet 3 for receiving treated lignocellulosic material from a reactor (not shown). The outlet section 4 is arranged at a downstream end of the discharge arrangement 1 and configured to receive lignocellulosic material from the screw conveyor section 2.

The screw conveyor section 2 comprises an essentially tubular side wall 2a that defines an inner space. A discharge screw 5 is rotatably mounted within the inner space for transport of lignocellulosic material in a material transport direction A from the inlet 3 towards the outlet section 4. The discharge screw 5 comprises a central shaft 5a arranged to be rotated around a rotational axis by means of a drive unit 6. A helical screw flight 5b extends along a portion of the length of the central shaft 5a.

The outlet section 4 comprises an essentially tubular side wall 4a that defines an inner space. A first outlet 7a and a second outlet 7b (not shown) are arranged in the side wall to allow lignocellulosic material to be discharged from the outlet section 4. A material spreader 8 is rotatably mounted within the inner space and configured to propel the material through the first and second outlets 7a,b. For this purpose, the material spreader 8 comprises a plurality of blades 8a-d connected to a rotatable central shaft 11 and extending radially outwards from the shaft 11 towards the side wall 4a. In this embodiment, four blades 8a-d distributed along a circumference of the material spreader 8 with 90 degrees interval. In other embodiments, the material spreader may comprise any suitable number of blades. The shaft 11 is rotated around a rotational axis of the material spreader by a drive unit 9. In this embodiment, the rotational axis of the material spreader 8 is coaxial with the rotational axis of the conveyor screw 5.

The drive unit 9 is controlled by the control unit 10, which is configured to change the rotational direction of the material spreader 8 with certain intervals between a first rotational direction and an opposite second rotational direction. Each blade 8a comprises two opposite surfaces configured to engage with the lignocellulosic material depending on the current rotational direction of the material spreader 8. The surfaces of the blades 8a propel the lignocellulosic material radially outwards and in the first and second rotational directions towards the first and second outlets 7a,b.

The lignocellulosic material is received through the inlet 3 and transported by means of the discharge screw 5 through the screw conveyor section 4a to the outlet section 4, where the blades 8a of the material spreader 8 alternately propels the lignocellulosic material towards the first and second outlets 7a,b depending on the rotational direction of the material spreader 8. A first blow line (not shown) may be connected to the first outlet 7a and a second blow line (now shown) may be connected to the second outlet 7b. The first and second blow lines are arranged to receive the discharged lignocellulosic material and transport it to other equipment for further processing.

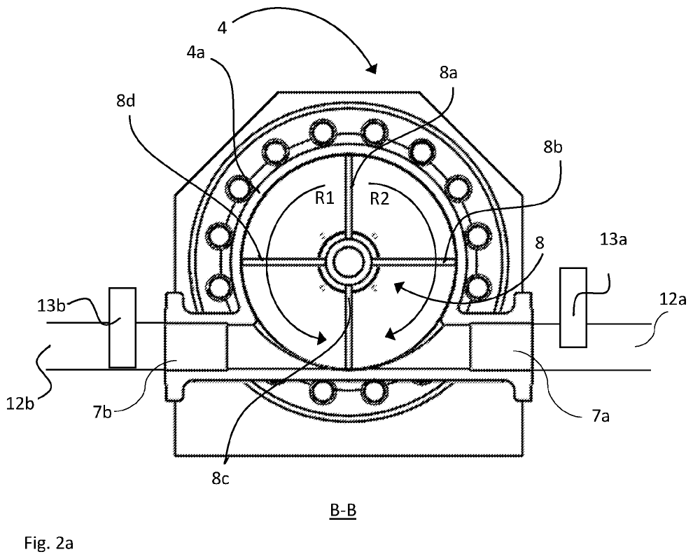

FIG. 2a shows a cross-sectional view through the outlet section along line BB in FIG. 1.

The first and second outlets 7a,b are located at a distance from one another along an inner circular perimeter of the side wall 4a. The first and second outlets 7a,b are arranged at opposite sides of the outlet section 4 seen in a transverse direction and in a lower portion of the outlet section 4 seen in a vertical direction. In other embodiments, the outlets 7a, b may be arranged in other ways (see FIG. 2b and FIG. 2c).

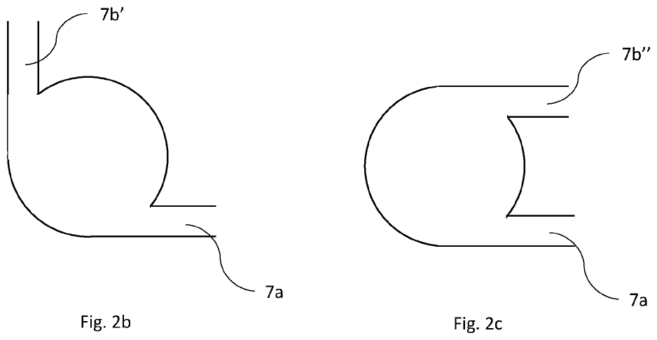

The rotation of the material spreader 8 is alternated between a first rotational direction R1 and a second rotational direction R2. When the material spreader 8 is rotated in the first rotational direction R1, the blades 8a move the lignocellulosic material through the first outlet 7a and into a first blow line 12a, and when the material spreader 8 is rotated in the second rotational direction R2, the blades 8a moves the lignocellulosic material through the second outlet 7 b and into a second blow line 12b. A first discharge (blow) valve 13a is located at the first outlet 7a and a second discharge (blow) valve 13b is located at the second outlet 7b. As will be explained below with reference to FIG. 3, the discharge valves 13a,b are controlled by means of the control unit control unit 10 shown in FIG. 1. The control unit is configured to order the first discharge valve 13a to (at least partially) open when the material spreader 8 is rotated in the first rotational direction R1 and lignocellulosic material is discharged through the first outlet 7a and to order the valve 13a to (at least partially) close when the material spreader 8 is rotated in the second rotational direction R2 and lignocellulosic material is discharged through the second outlet 7b. Correspondingly, the control system is configured to order the second discharge valve 13b to (at least partially) open when the material spreader 8 is rotated in the second rotational direction R2 and (at least partially) close the valve 13b when the material spreader 8 is rotated in the first rotational direction R1. In the embodiment shown in FIG. 2, the first discharge valve 13a is schematically shown as partially open to achieve a steam explosion effect in the first blow line 12a and the second discharge valve 13b is schematically shown as almost completely closed.

The first outlet 7a extends outwards through the side wall of the outlet section 4 substantially along a tangent to the first rotational direction R1 of the material spreader 8 and the second outlet 7b extends outwards through the side wall of the outlet section 8 substantially along a tangent to the second rotational direction R2 of the material spreader. This ensures that the lignocellulosic material, which is propelled outwards and along the first and second rotational directions R1, R2 is smoothly discharged through the first and second outlets 7a,b.

FIG. 2b shows an alternative embodiment of the outlet section of a discharge arrangement according to the invention. Herein, the first outlet 7a is arranged in the same way as in FIG. 2a whereas the second outlet 7b′ is arranged on the opposite side of the outlet section 4b seen in the transverse direction and in an upper portion of the outlet section 4b seen in the vertical direction. The second outlet 7b′ extends outwards through the side wall of the outlet section 8 substantially along a tangent to the second rotational direction R2 of the material spreader.

FIG. 2c shows an alternative embodiment of the outlet section of a discharge arrangement according to the invention. Herein, the first outlet 7a is arranged in the same way as in FIG. 2a whereas the second outlet 7b“ is arranged on the same side of the outlet section 4b seen in the transverse direction and in an upper portion of the outlet section 4b seen in the vertical direction. The second outlet 7b” extends outwards through the side wall of the outlet section 8 substantially along a tangent to the second rotational direction R2 of the material spreader.

FIG. 3 is a schematic illustration of an embodiment of a discharge system 14 according to the invention. The discharge system is shown along with a pre-hydrolysis treatment reactor 17 and a blow tank 15 which together constitute an embodiment of a treatment system according to the invention.

The treatment reactor 17 comprises an internal conveyor screw 17a rotatably arranged in the reactor vessel, the internal conveyor screw being configured to mechanically transport the lignocellulosic material through the reactor vessel in a conveying direction along a rotational axis of the internal conveyor screw and out through an outlet 17b of the reactor, the outlet being disposed at a downstream end of the reactor. The reactor is a substantially vertically arranged reactor vessel, wherein the discharge screw arrangement is located vertically below the reactor vessel at the downstream end of the reactor vessel, such that the lignocellulosic material can fall down vertically from the outlet 17b of the reactor vessel into inlet 3 of the discharge screw arrangement.

The discharge system 14 comprises a discharge arrangement 1 as shown in FIGS. 1 and 2a.

The control unit is electrically connected with the valves 13a, 13b and the drive unit 9 for control thereof (as explained above with reference to FIGS. 1 and 2a).

The discharge valves 13a, 13ab are connected via respective blow lines 12a, 12b to blow tank 15 in which the steam exploded lignocellulosic material along with blow steam is received.

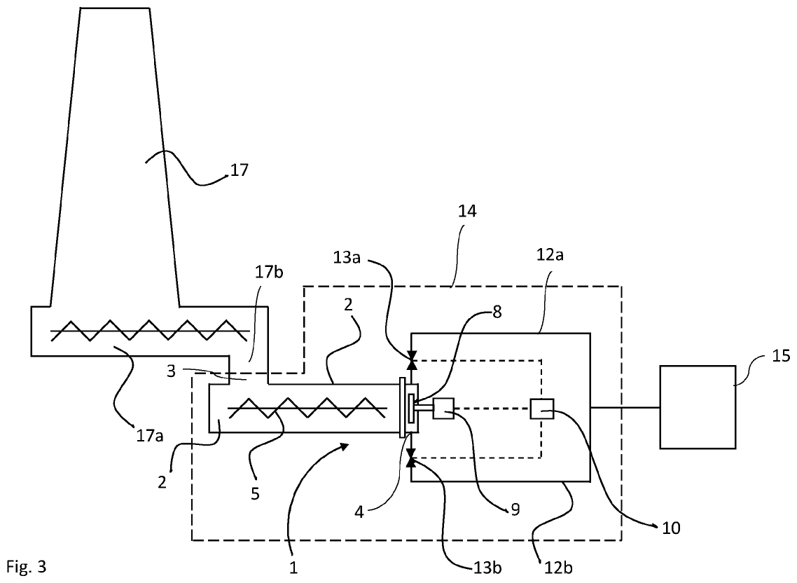

FIG. 4 is a schematic illustration of an alternative embodiment of a discharge system according to the invention.

The discharge system 114 corresponds to the discharge system 14 shown in FIG. 3 in that it comprises an outlet section 104, a control unit 110, discharge valves 113a-b, a blow tank 115 and blow lines 112a, 112b corresponding to ref. 10, 13 a-b, 15 and 12 a-b in FIG. 3.

The embodiment in FIG. 4 however differs from the embodiment in FIG. 3 in that the blow lines 112a and 112b are each provided with two steam valves. Steam valve 119al is arranged to provide steam into the first outlet of the outlet section and steam valve 119a2 is arranged to provide steam into the first blow line 112a (downstream of the first discharge valve 113 a). Steam valve 119 b1 is arranged to provide steam into the second outlet of the outlet section and steam valve 119 b2 is arranged to provide steam into the second blow line 112b (downstream of the first discharge valve 113b). An additional difference is that the control unit 110 is further configured to control the steam valves 119a1, 119a2, 119b1 and 119b2. The steam valves are connected via respective steam lines 116a, 116b to a source of steam 118.

The control unit 110 is configured to, when the material spreader rotates in the first rotational direction R1, control steam valve 119al and 119a2 to be closed, and to control steam valve 119b1 and 119b2 such as to direct a larger portion of the steam flow, suitably about 85-95% of the steam, into the second outlet (7b, see FIG. 2a) and a smaller portion of the steam flow, suitably 5-15%, in a direction away from the outlet section to preheat a downstream portion of the blow line 112b before a change in rotational direction of the material spreader.

Correspondingly, when the material spreader rotates in the second rotational direction R2, the control unit 110 is configured to control steam valves 119b1 and 119b2 to be closed, and control steam valves 119a1 and 119a2 such as to direct a larger portion of the steam flow, suitably about 85-95% of the steam, into the first outlet (7a, see FIG. 2a) and a smaller portion of the steam flow, suitably 5-15%, in a direction away from the outlet section to preheat a downstream portion of the blow line 112a before a change in rotational direction of the material spreader.

Other embodiments correspond to the embodiment in FIG. 4 except that the steam valves 119a1, 119a2 and/or 119b1, 119b2 is/are replaced by a respective two-way valve.

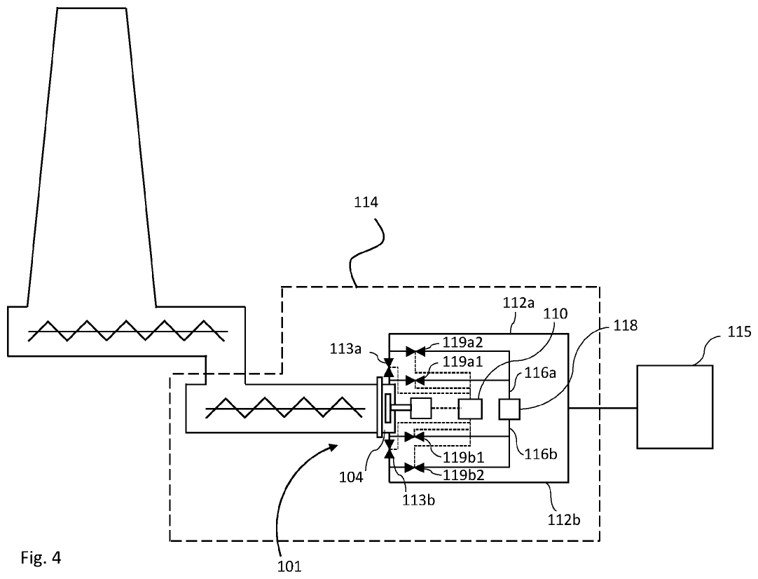

FIG. 5 is a schematic illustration of the material spreader of the discharge arrangement in FIG. 1. As can be seen in the figure, the material spreader comprises four radially extending blades (8a for example) angularly distributed around the circumference of the material spreader at 90 degrees angular interval from each other. The blades each comprises a flat/plane first surface 20a configured to transport the material in the first rotational direction R1 and an opposite flat/plane second surface 20b configured to transport the material in the second rotational direction R2. The blades and the first/second surfaces thereof extend orthogonally relative to a rotational plane orthogonal to the rotational axis of the material spreader.

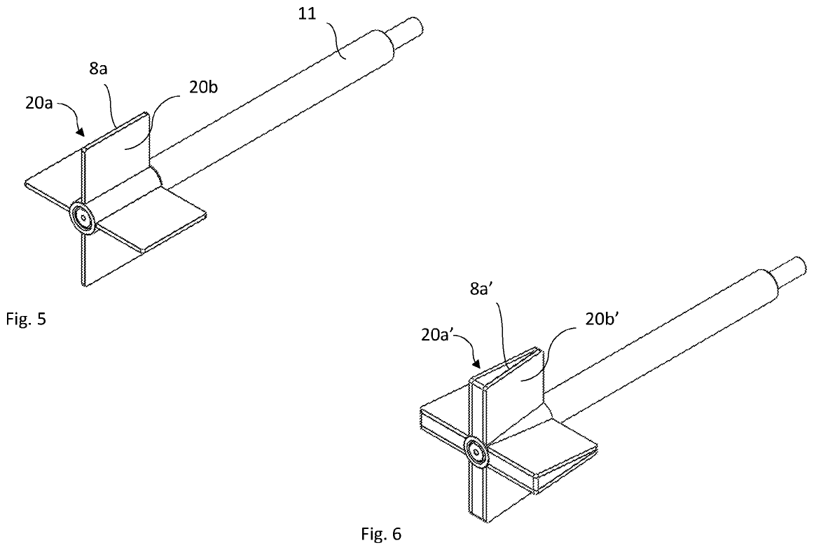

FIG. 6 is a schematic illustration of an alternative embodiment of a material spreader of a discharge system according to the invention. The embodiment in FIG. 6 corresponds to the embodiment in FIG. 5 except that the flat/plane first and second surfaces 20a′, 20b′ are disposed with an acute angle relative the rotational axis of the material spreader, so that the blades have increasing thickness in the material transport direction.

The description above and the appended drawings are to be considered as non-limiting examples of the invention. The person skilled in the art realizes that several changes and modifications may be made within the scope of the invention. For example, the material spreader shown in FIG. 6 can be applied in the embodiments shown in FIG. 1-4. Further, the steam valves 119a2 and 119b2 shown in FIG. 4 may in other embodiments be omitted such that the whole steam flow is directed into the respective outlet.

1-15. (canceled)

16. A discharge arrangement for discharging lignocellulosic material, said discharge arrangement comprising:
a screw conveyor section comprising an inlet for receiving lignocellulosic material;
an outlet section configured to receive lignocellulosic material from the screw conveyor section;
a conveyor screw rotatably arranged within the screw conveyor section for transporting the material in a material transport direction from the inlet to the outlet section;
a material spreader rotatably mounted within the outlet section, wherein the material spreader is rotatable in a first rotational direction and an opposite second rotational direction around a rotational axis extending in the material transport direction, which material spreader comprises at least one blade configured to rotate around the rotational axis to transport the lignocellulosic material radially outwards and in the first and second rotational directions during rotation of the material spreader;
a drive unit configured to rotate the material spreader around the rotational axis; and
a first outlet and a second outlet arranged in a side wall of the outlet section, said first and second outlets being arranged at a distance from one another along an inner perimeter of the side wall, said first outlet being arranged to receive material transported by the blade in the first rotational direction and the second outlet being arranged to receive material transported by the blade in the second rotational direction.

17. The discharge arrangement according to claim 16, wherein the first outlet extends outwards substantially along a tangent to the first rotational direction of the material spreader and/or the second outlet extends outwards substantially along a tangent to the second rotational direction of the material spreader.

18. The discharge arrangement according to claim 16, wherein said at least one blade comprises a first surface configured to transport the material in the first rotational direction and an opposite second surface configured to transport the material in the second rotational direction.

19. The discharge arrangement according to claim 18, wherein at least one of the first and second surfaces is at least partly disposed orthogonal to a rotational plane of the blade.

20. The discharge arrangement according to claim 18, wherein at least one of the first and second surfaces is at least partly disposed with an acute angle relative the rotational axis of the material spreader, so that the at least one blade has an increasing thickness in the material transport direction.

21. The discharge arrangement according to claim 16, further comprising a first discharge valve configured to regulate the flow of material through the first outlet and a second discharge valve configured to regulate the flow of material through the second outlet.

22. The discharge arrangement according to claim 16, further comprising a first steam line configured to transport steam from a steam source towards the first outlet and/or a second steam line configured to transport steam from a steam source towards the second outlet, said discharge arrangement further comprising at least one steam valve configured to regulate the flow of steam through the first outlet and/or at least one steam valve configured to regulate the flow of steam through the second outlet.

23. A discharge system for transport of lignocellulosic material, said discharge system comprising:
a discharge arrangement according to claim 16; and
a control unit configured to control the drive unit to alternate the rotational direction of the material spreader between the first and second rotational directions.

24. The discharge system according to claim 23, wherein:
the discharge arrangement comprises a first discharge valve configured to regulate the flow of material through the first outlet and a second discharge valve configured to regulate the flow of material through the second outlet; and
said control unit is configured to control the first and second discharge valves.

25. The discharge system according to claim 23, wherein:
the discharge arrangement further comprises a first steam line configured to transport steam from a steam source towards the first outlet and/or a second steam line configured to transport steam from a steam source towards the second outlet, said discharge arrangement further comprising at least one steam valve configured to regulate the flow of steam through the first outlet and/or at least one steam valve configured to regulate the flow of steam through the second outlet; and
said control unit is configured to control one or more of said steam valves.

26. A method comprising:
providing a discharge arrangement for discharging lignocellulosic material, said discharge arrangement comprising:
a screw conveyor section comprising an inlet for receiving lignocellulosic material;
an outlet section configured to receive lignocellulosic material from the screw conveyor section;
a conveyor screw rotatably arranged within the screw conveyor section for transporting the material in a material transport direction from the inlet to the outlet section;
a material spreader rotatably mounted within the outlet section, wherein the material spreader is rotatable in a first rotational direction and an opposite second rotational direction around a rotational axis extending in the material transport direction, which material spreader comprises at least one blade configured to rotate around the rotational axis to transport the lignocellulosic material radially outwards and in the first and second rotational directions during rotation of the material spreader;
a drive unit configured to rotate the material spreader around the rotational axis; and
a first outlet and a second outlet arranged in a side wall of the outlet section, said first and second outlets being arranged at a distance from one another along an inner perimeter of the side wall, said first outlet being arranged to receive material transported by the blade in the first rotational direction and the second outlet being arranged to receive material transported by the blade in the second rotational direction;
transporting the material towards the material spreader by means of the conveyor screw; and
using a control unit, controlling the drive unit to alternately rotate the material spreader in the first and second rotational directions, so that said material is alternately propelled out of the outlet section through the first and second outlets.

27. The method according to claim 26, comprising opening a first discharge valve configured to regulate the flow of material through the first outlet when the material spreader is rotated in the first rotational direction and opening a second discharge valve configured to regulate the flow of material through the second outlet when the material spreader is rotated in the second rotational direction.

28. The method according to claim 27, comprising closing the second discharge valve when the material spreader is rotated in the first rotational direction and closing the first discharge valve when the material spreader is rotated in the second rotational direction.

29. The method according to claim 26, comprising controlling at least one steam valve configured to control a flow of steam through the first outlet, and at least one steam valve configured to control a flow of steam through the second outlet, so that steam is injected through the first outlet when the material spreader is rotated in the second rotational direction and through the second outlet when the material steam is rotated in the first rotational direction.
